(19) United States
(12) Patent Application Publication
(10) Pub. No.: US 2026/0269076 A1
(43) Pub. Date: Sep. 10, 2026

(54) SYSTEM

(71) Applicant: SOFTBANK GROUP CORP., Tokyo (JP)

(72) Inventor: Yasunori KANEMITSU, Tokyo (JP)

(73) Assignee: SOFTBANK GROUP CORP., Tokyo (JP)

(21) Appl. No.: 19/557,215

(22) Filed: Mar. 5, 2026

Related U.S. Application Data
(60) Provisional application No. 63/767,922, filed on Mar. 6, 2025.

(51) Int. Cl. G16H 50/30; A61B 5/01; A61B 5/024; A61B 5/145; G06N 3/04; G10L 15/22

(52) U.S. Cl. CPC G16H 50/30; A61B 5/01; A61B 5/024; A61B 5/14542; G06N 3/04; G10L 15/22

(57) ABSTRACT

A system that supports user health management using wearable devices, terminals, and cloud servers is disclosed. The wearable device collects biometric data such as heart rate, body temperature, activity level, and sleep patterns in real time and transmits it to the cloud server via the terminal. A generative AI installed on the cloud server analyzes the data using machine learning algorithms to evaluate stress levels and sleep quality. Analysis results are provided to the user via an application on the terminal, offering specific health advice. By implementing the advice and providing feedback on the results, the AI can deliver more personalized advice. This system enables users to monitor their health status in real time during daily life and improve their quality of life.

### CROSS-REFERENCE TO RELATED APPLICATION

This application claims priority under 35 U.S.C. § 119 to U.S. Provisional Patent Application No. 63/767,922, filed on Mar. 6, 2025, the entire contents of which are incorporated herein by reference.

### BACKGROUND

### Technical Field

The present disclosure relates to a system.

### Related Art

Japanese Patent Application Publication Laid-Open (JP-A) No. 2022-180282 discloses a persona chatbot control method performed by at least one processor, comprising: a step of receiving a user utterance; a step of adding to the user utterance a prompt containing a description of the chatbot's persona and related instructions; a step of encoding the prompt; and a step of inputting the encoded prompt into a language model to generate a chatbot utterance in response to the user utterance.

### SUMMARY

A system and method for solving the effort required for data input when using generative AI and the lack of real-time support in users' daily lives is provided. Conventional generative AI requires users to manually input data, a process that is cumbersome and time-consuming, placing a significant burden on users. Furthermore, it is difficult for users to receive appropriate advice at the exact moment they need it. Particularly in health management, real-time data analysis and advice provision are essential. This invention aims to solve these problems by continuously collecting biometric data using wearable devices and providing appropriate advice when users need it through automatic analysis on cloud servers.

Furthermore, anticipating the future proliferation of devices capable of reading thoughts, it aims to achieve a deeper level of user understanding and personalized support. This enables improved health management and quality of life for users while providing efficient and enriched lifestyles for both corporations and consumers.

As a means to solve the challenges, a system comprising: a data collection unit that collects biometric data from a wearable device; a data transmission unit that sends the collected biometric data to a cloud server; a data analysis unit that includes generative AI for analyzing the biometric data on the cloud server; and a user interaction unit that provides advice to the user based on the analysis results is provided. With this system, users need only wear a wearable device for their biometric data—such as heart rate, body temperature, activity levels, and sleep patterns—to be collected in real time. This data is transmitted to the cloud server via the data transmission unit and analyzed using machine learning algorithms within the data analysis unit. The analysis results are provided to the user through the user interaction unit, enabling the user to receive appropriate advice at the necessary time. This reduces the burden of data input and enables real-time health management and improvement in quality of life.

### BRIEF DESCRIPTION OF THE DRAWING

FIG. 1 is a conceptual diagram showing an example configuration of the data processing system according to the first embodiment.

FIG. 2 is a conceptual diagram showing an example of key functional components of the data processing device and smart device according to the first embodiment.

FIG. 3 is a conceptual diagram showing an example configuration of the data processing system according to the second embodiment.

FIG. 4 is a conceptual diagram showing an example of the main functions of the data processing device and smart glasses according to the second embodiment.

FIG. 5 is a conceptual diagram showing an example configuration of a data processing system according to a third embodiment.

FIG. 6 is a conceptual diagram showing an example of the main functions of the data processing device and headset-type terminal according to the third embodiment.

FIG. 7 is a conceptual diagram showing an example configuration of a data processing system according to the fourth embodiment.

FIG. 8 is a conceptual diagram showing an example of the main functions of the data processing device and robot according to the fourth embodiment.

FIG. 9 shows an emotion map onto which multiple emotions are mapped.

FIG. 10 shows an emotion map onto which multiple emotions are mapped.

### DETAILED DESCRIPTION

The following describes an example embodiment of a system according to the present disclosure with reference to the accompanying drawings.

First, the terminology used in the following description is explained.

In the following embodiments, a processor (hereinafter simply referred to as a “processor”) may be a single arithmetic unit or a combination of multiple arithmetic units. Furthermore, the processor may be a single type of arithmetic unit or a combination of multiple types of arithmetic units. Examples of processing units include a CPU (Central Processing Unit), a GPU (Graphics Processing Unit), a GPGPU (General-Purpose Computing on Graphics Processing Units), an APU (Accelerated Processing Unit), and the like.

In the following embodiments, signed RAM (Random Access Memory) is a memory where information is temporarily stored and is used as working memory by the processor.

In the following embodiments, the signed storage is one or more non-volatile storage devices that store various programs and various parameters, etc. Examples of non-volatile storage devices include flash memory (SSD (Solid State Drive)), magnetic disks (e.g., hard disk), or magnetic tape.

In the following embodiments, the communication I/F (Interface) is an interface that includes a communication processor and an antenna, among other components. The communication I/F governs communication between multiple computers. Examples of communication standards applicable to the communication I/F include wireless communication standards such as 5G (5 th Generation Mobile Communication System), Wi-Fi (registered trademark), or Bluetooth (registered trademark).

In the following embodiments, “A and/or B” is synonymous with “at least one of A and B.” That is, “A and/or B” may mean A alone, B alone, or a combination of A and B. Furthermore, in this specification, when three or more items are connected using “and/or,” the same concept applies as for “A and/or B”.

### First Embodiment

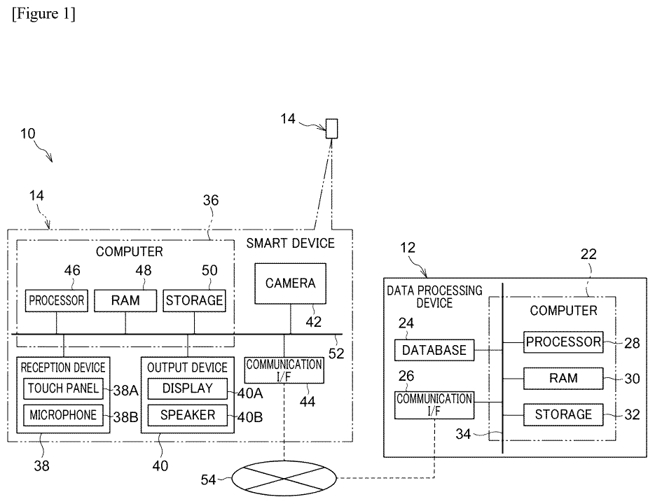

FIG. 1 shows an example configuration of a data processing system 10 according to the first embodiment.

As shown in FIG. 1, the data processing system 10 includes a data processing device 12 and a smart device 14. An example of the data processing device 12 is a server.

The data processing device 12 includes a computer 22, a database 24, and a communication I/F 26. The computer 22 is an example of a “computer” according to the technology of the present disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 include a WAN (Wide Area Network) and/or a LAN (Local Area Network).

The smart device 14 includes a computer 36, a reception device 38, an output device 40, a camera 42, and a communication I/F 44. The computer 36 includes a processor 46, RAM 48, and storage 50. The processor 46, RAM 48, and storage 50 are connected to a bus 52. The reception device 38, output device 40, and camera 42 are also connected to the bus 52. The reception device 38, output device 40, and camera 42 are also connected to the bus 52.

The reception device 38 includes a touch panel 38A and a microphone 38B, among other components, and receives user input. The touch panel 38A receives user input via contact with an indicator (e.g., a pen or finger) by detecting such contact. The microphone 38B receives voice-based user input by detecting the user's voice. The control unit 46A transmits data indicating the user input received via the touch panel 38A and microphone 38B to the data processing device 12. Within the data processing device 12, the specific processing unit 290 acquires the data indicating the user input.

Output device 40 includes display 40A and speaker 40B, presenting data to user 20 by outputting it in a perceptible form (e.g., audio and/or text). Display 40A displays visual information such as text and images according to instructions from processor 46. Speaker 40B outputs audio according to instructions from processor 46. Camera 42 is a compact digital camera equipped with an optical system including a lens, aperture, and shutter, and an imaging element such as a CMOS (Complementary Metal-Oxide-Semiconductor) image sensor or a CCD (Charge Coupled Device) image sensor.

The communication interface 44 is connected to the network 54. The communication interfaces 44 and 26 manage the exchange of various information between processor 46 and processor 28 via network 54.

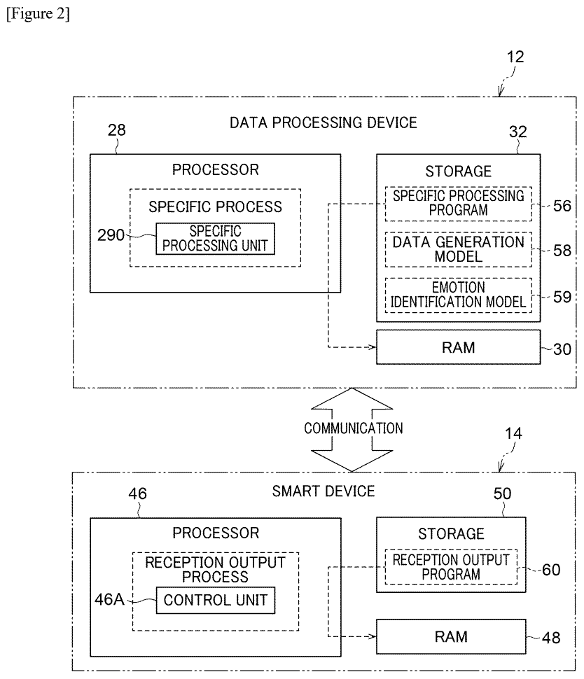

FIG. 2 shows an example of the main functions of the data processing device 12 and the smart device 14.

As shown in FIG. 2, specific processing is performed by processor 28 in data processing device 12. Specific processing program 56 is stored in storage 32. Specific processing program 56 is an example of a “program” related to the technology of this disclosure. Processor 28 reads the specific processing program 56 from the storage 32 and executes the read specific processing program 56 on the RAM 30. The specific processing is realized by the processor 28 operating as a specific processing unit 290 according to the specific processing program 56 executed on the RAM 30.

Storage 32 stores a data generation model 58 and an emotion identification model 59. The data generation model 58 and emotion identification model 59 are used by specific processing unit 290. Specific processing unit 290 can estimate a user's emotion using emotion identification model 59 and perform specific processing using the user's emotion. The emotion estimation function (emotion identification function) using the emotion identification model 59 performs various estimations and predictions concerning the user's emotion, including estimation and prediction of the user's emotion, but is not limited to such examples. Furthermore, estimation and prediction of emotion also includes, for example, analysis (parsing) of emotion.

The smart device 14 performs reception output processing via the processor 46. The reception output program 60 is stored in the storage 50. The reception output program 60 is used in conjunction with the specific processing program 56 by the data processing system 10. The processor 46 reads the reception output program 60 from the storage 50 and executes the read reception output program 60 on the RAM 48. The specific processing is performed by the processor 46 operating as a control unit 46A according to the specific processing program 60 executed on the RAM 48. Note that the smart device 14 may also have data generation models and emotion identification models similar to the data generation model 58 and emotion identification model 59, and may perform processing similar to that of the specific processing unit 290 using these models. The reception output processing is realized by the processor 46 operating as the control unit 46A according to the reception output program 60 executed on the RAM 48.

Other devices besides the data processing device 12 may also have the data generation model 58. For example, a server device (e.g., a generation server) may have the data generation model 58. In this case, the data processing device 12 communicates with the server device having the data generation model 58 to obtain processing results (such as prediction results) obtained using the data generation model 58. Furthermore, the data processing device 12 may be the server device itself, or it may be a terminal device owned by a user (e.g., a mobile phone, robot, home appliance, etc.). Next, an example of processing by the data processing system 10 according to the first embodiment will be described.

### Example 1

The flow of specific processing in Example 1 is described below. The components of the system described below are implemented by the data processing device 12 and the smart device 14. The data processing device 12 is referred to as the “server,” and the smart device 14 is referred to as the “terminal.”

### Implementation Examples for Carrying Out the Invention

The embodiment for implementing the present invention will now be described in further detail. The present invention is a system configured using a wearable device, a terminal, and a cloud server. The following details how each function is realized.

First, the wearable device is described. This device is worn on the user's body and collects biometric data such as heart rate, body temperature, blood pressure, activity level, and sleep patterns in real time. Specifically, it incorporates sensors including a heart rate sensor, temperature sensor, accelerometer, gyroscope, and optical sensor, which work together to acquire data. For example, the heart rate sensor detects changes in blood flow using optical technology to measure heart rate. Additionally, the accelerometer and gyroscope detect the user's movement and posture to measure activity levels. This data is temporarily stored in the device's memory.

Next, the terminal is described. The terminal is a portable electronic device, such as a smartphone or tablet, that communicates with the wearable device via Bluetooth or Wi-Fi. The terminal is equipped with a data transmission unit responsible for sending data collected from the wearable device to a cloud server. The data transmission unit temporarily buffers the data and efficiently transmits it when the communication environment is ready. For example, it has the capability to automatically upload data when the user connects to a Wi-Fi environment. Furthermore, a dedicated application is installed on the terminal, allowing the user to check data and receive advice from the AI through the app.

The cloud server is described below. The cloud server includes a data analysis unit where generative AI operates. The data analysis unit uses machine learning algorithms to analyze transmitted biometric data. Specifically, the AI performs pattern recognition based on large datasets to detect anomalies and trends. For example, it estimates stress levels from heart rate variability to assess the user's health status. It also analyzes sleep data to quantify sleep quality and provide specific suggestions for improvement. Furthermore, the AI monitors changes in the user's health status by comparing it to historical data, supporting long-term health management.

The user interaction component is described. This component is implemented as an application on the device, providing advice to the user based on the analysis results. Specifically, the app visually displays the analysis results to enable intuitive user understanding. For example, when stress levels are high, it provides guidance on relaxation breathing techniques or meditation. When sleep quality is poor, it suggests improvements to the sleep environment or bedtime routines. Users can execute the advice through the app and provide feedback on the results. This feedback is utilized as data for the AI to learn from, helping it provides more personalized advice.

Furthermore, anticipating the future widespread adoption of mind-reading devices, plans are in place to transmit data obtained from these devices to cloud servers for AI analysis. This will enable a deeper understanding of users' emotions and thought patterns, allowing for the provision of even more personalized support. For example, by analyzing a user's thought patterns, it will be possible to identify the causes of stress and propose specific countermeasures.

Thus, the present invention is a system that combines wearable devices, terminals, and cloud servers to realize user health management and improved quality of life, aiming to provide efficient and enriched lifestyles for corporations and consumers.

### System Configuration

The system according to this embodiment comprises a data collection unit, a data transmission unit, a data analysis unit, and a user interaction unit.

The data collection unit is mounted on a wearable device and is responsible for collecting the user's biometric data in real time. This unit incorporates various sensors, such as a heart rate sensor, a temperature sensor, an accelerometer, a gyroscope, and an optical sensor. These sensors work together to acquire data. For example, the heart rate sensor uses optical technology to detect changes in blood flow and measure heart rate. The temperature sensor measures the user's skin temperature, enabling real-time monitoring of body temperature fluctuations. The accelerometer and gyroscope detect the user's movement and posture, measuring activity levels. The optical sensor is used to measure blood oxygen saturation, allowing for a more detailed assessment of the user's health status.

The data transmission unit is installed in the terminal and is responsible for sending data collected from the wearable device to the cloud server. This unit communicates with the wearable device using Bluetooth or Wi-Fi, temporarily buffers the data, and efficiently transmits it when the communication environment is ready. For example, it has a function to automatically upload data when the user connects to a Wi-Fi environment. Furthermore, the data transmission unit can adjust the frequency and timing of data transmission according to user settings. Additionally, it incorporates data encryption functionality to ensure the security of data during transmission.

The data analysis unit is located on the cloud server and operates generative AI. This unit analyzes transmitted biometric data using machine learning algorithms. Specifically, the AI performs pattern recognition based on large datasets to detect anomalies and trends. For example, it estimates stress levels from heart rate variability to assess the user's health status. It also analyzes sleep data to quantify sleep quality and provide specific recommendations for improvement. Furthermore, the AI monitors changes in the user's health status by comparing it to historical data, supporting long-term health management. Specific examples of prompt sentences fed to the generative AI include: “Analyze the user's heart rate data and estimate their stress level” or “Evaluate sleep quality based on sleep data and propose improvement measures.”

The User Interaction Unit is implemented as an application on the device and provides advice to the user based on the analysis results. This unit visually displays the analysis results to enable intuitive user understanding. For example, if stress levels are high, it provides guidance on relaxation breathing techniques or meditation. If sleep quality is poor, it suggests improvements to the sleep environment or bedtime routines. Users can execute the advice through the app and provide feedback on the results. This feedback is utilized as data for the AI to learn from, helping it provides more personalized advice. Furthermore, the User Interaction Unit includes a voice assistant function, allowing users to call the AI via voice and receive advice.

Thus, the system according to this embodiment combines a data collection unit, a data transmission unit, a data analysis unit, and a user interaction unit to realize user health management and improved quality of life. It aims to provide efficient and enriched lifestyles for corporations and consumers.

### Implementation Steps

### Step 1: Data Collection

The user wears a wearable device to collect real-time biometric data such as heart rate, body temperature, activity levels, and sleep patterns during daily life. This device incorporates sensors including a heart rate sensor, temperature sensor, accelerometer, gyroscope, and optical sensor, which work together to acquire data. For example, the heart rate sensor uses optical technology to detect changes in blood flow and measure heart rate. The temperature sensor measures the user's skin temperature, enabling real-time monitoring of body temperature fluctuations. The accelerometer and gyroscope detect the user's movement and posture to measure activity levels. The optical sensor is used to measure blood oxygen saturation, enabling a more detailed assessment of the user's health status.

### Step 2: Data Transmission

Collected data is transmitted to a cloud server via the data transmission unit installed in the terminal device. The terminal device is a portable electronic device such as a smartphone or tablet, communicating with the wearable device using Bluetooth or Wi-Fi. The data transmission unit temporarily buffers the data and efficiently transmits it when the communication environment is ready. For example, it has a function to automatically upload data when the user connects to a Wi-Fi environment. Furthermore, the data transmission unit can adjust the frequency and timing of data transmission according to the user's settings. Additionally, it is equipped with data encryption functionality to ensure the security of data during transmission.

### Step 3: Data Analysis

Generative AI operates within the data analysis unit established on the cloud server to analyze transmitted biometric data. This unit analyzes the transmitted biometric data using machine learning algorithms. Specifically, the AI performs pattern recognition based on large datasets to detect anomalies and trends. For example, it estimates stress levels from heart rate variability to assess the user's health status. It also analyzes sleep data to quantify sleep quality and provide specific improvement suggestions. Furthermore, the AI monitors changes in the user's health status by comparing it with historical data, supporting long-term health management. Specific examples of prompt sentences fed to the generative AI include: “Analyze the user's heart rate data and estimate their stress level,” and “Evaluate sleep quality based on sleep data and propose improvement measures.”

### Step 4: User Interaction

Analysis results are provided to the user through the user interaction component, implemented as an application on the device. This component visually displays the analysis results, enabling the user to understand them intuitively. For example, if stress levels are high, it provides guidance on relaxation breathing techniques or meditation. If sleep quality is poor, it suggests improvements to the sleep environment or bedtime routines. Users can execute the advice through the app and provide feedback on the results. This feedback is utilized as data for the AI to learn from, helping it provides more personalized advice. Furthermore, the user interaction component includes a voice assistant function, allowing users to call upon the AI via voice commands to receive advice.

### Specific Use Cases

Consider a user aiming for health management utilizing the present system. This user routinely wears a wearable device, collecting real-time biometric data such as heart rate, body temperature, activity levels, and sleep patterns. The heart rate sensor continuously monitors the user's heart rate to detect fluctuations in stress levels. The temperature sensor captures subtle changes in body temperature to detect health abnormalities early. The accelerometer and gyroscope record the user's movement and posture to assess daily activity levels. The optical sensor measures blood oxygen saturation and monitors respiratory status.

The collected data is transmitted to a cloud server via the terminal. The data transmission unit efficiently buffers the data and automatically sends it to the cloud server when the communication environment is established. In the data analysis unit established on the cloud server, generative AI analyzes the data using machine learning algorithms. The AI estimates stress levels from heart rate variability and evaluates sleep quality based on sleep data. Furthermore, it monitors changes in the user's health status by comparing with past data, supporting long-term health management.

Analysis results are provided to the user through the user interaction unit. This unit visually displays the analysis results, enabling the user to understand them intuitively. For example, when stress levels are high, it provides guidance on breathing techniques or meditation for relaxation. When sleep quality is poor, it suggests improvements to the sleep environment or bedtime routines. Users can implement the advice through the app and provide feedback on the results. This feedback is utilized as data for AI learning, helping to provide more personalized advice.

Examples of prompts fed to the generative AI include: “Analyze the user's heart rate data and estimate their stress level” or “Evaluate sleep quality based on sleep data and propose improvement strategies.” This enables users to monitor their health status in real-time during daily life and receive appropriate advice.

### Application Example 1

The flow of specific processing in Application Example 1 is described below. The components of the system described below are implemented by the data processing device 12 and the smart device 14. The data processing device 12 is referred to as the “server,” and the smart device 14 is referred to as the “terminal.”

### Implementation Examples for the Present Invention

The present invention is a system for the care sector that uses wearable devices, terminals, and cloud servers to monitor the health status of elderly individuals and those requiring care in real time and provide appropriate care support.

First, a wearable device is attached to the body of the care recipient. This device has the capability to collect real-time biometric data such as heart rate, body temperature, activity level, sleep patterns, and blood oxygen saturation. Specifically, a heart rate sensor uses optical technology to detect changes in blood flow and measure heart rate. For example, if the heart rate is abnormally high even when the care recipient is at rest, this may indicate stress or poor physical condition. The temperature sensor measures skin temperature, allowing real-time monitoring of subtle fluctuations in body temperature. For example, it can detect early signs of fever, reducing the risk of infection. Accelerometers and gyroscopes detect the care recipient's movements and posture, measuring activity levels. For example, they can assess the risk of falls and enable preventive measures. The optical sensor measures blood oxygen saturation and monitors respiratory status. For example, it can detect early signs of respiratory distress and prompt appropriate action.

Next, the collected data is transmitted to a cloud server via the care recipient's terminal. The terminal is a portable electronic device, such as a smartphone or tablet, which communicates with the wearable device using Bluetooth or Wi-Fi. The data transmission unit temporarily buffers the data and efficiently transmits it when the communication environment is ready. For example, it has a function to automatically upload data when connected to a Wi-Fi environment. It also incorporates data encryption functionality to ensure the security of data during transmission.

The cloud server includes a data analysis unit where generative AI operates. This unit analyzes transmitted biometric data using machine learning algorithms. Specifically, the AI performs pattern recognition based on large datasets to detect anomalies and trends. For instance, it estimates stress levels from heart rate variability and detects signs of fever from body temperature changes. It also analyzes sleep data to evaluate sleep quality and provides specific recommendations for improvement. Furthermore, the AI monitors changes in health status by comparing with past data and sends alerts to caregivers or medical staff when abnormalities are detected.

Analysis results are notified to the caregiver's device, proposing specific care plans or emergency response measures. For example, if a sudden fluctuation in heart rate is detected, the AI suggests the possibility of stress or physical discomfort, prompting the caregiver to implement relaxation techniques or contact a medical institution. If sleep quality is declining, it proposes improvements to the sleep environment or bedtime routines. The user interaction unit visually displays the analysis results, enabling caregivers to understand them intuitively. Furthermore, by receiving feedback on the results of the caregiver implementing the advice, the AI learns its effectiveness and can provide more personalized advice.

Thus, the present invention is a system that enables caregivers to grasp the health status of care recipients in real time and provide appropriate care by combining wearable devices, terminals, and cloud servers. This is expected to improve the quality of care and reduce the burden on caregivers. Furthermore, the care recipient themselves can check their health status and receive advice to improve their quality of life. For example, as part of daily health management, the care recipient can understand their own health status and improve their lifestyle habits as needed. Additionally, caregivers can monitor the care recipient's health status in real time, enabling them to respond quickly in emergencies. This improves the quality of care and ensures the safety of the care recipient.

### System Configuration

The system according to this embodiment comprises a data collection unit, a data transmission unit, a data analysis unit, and a user interaction unit.

The data collection unit is mounted on a wearable device and is responsible for collecting the care recipient's physiological data in real time. This unit incorporates various sensors, such as a heart rate sensor, temperature sensor, accelerometer, gyroscope, and optical sensor, which work together to acquire data. For example, the heart rate sensor uses optical technology to detect changes in blood flow and measure heart rate. This allows it to suggest the possibility of stress or poor health if the heart rate is abnormally high even when the care recipient is at rest. The temperature sensor measures skin temperature, enabling real-time monitoring of subtle fluctuations in body temperature. This facilitates early detection of signs of fever, reducing the risk of infection. Accelerometers and gyroscopes detect the care recipient's movements and posture to measure activity levels. This enables assessment of fall risk and implementation of preventive measures. Optical sensors measure blood oxygen saturation to monitor respiratory status. This allows early detection of signs of respiratory distress and prompts appropriate intervention.

The data transmission unit is installed in the terminal and is responsible for sending data collected from the wearable device to the cloud server. This unit communicates with the wearable device via Bluetooth or Wi-Fi, temporarily buffers the data, and efficiently transmits it when the communication environment is ready. For example, it has the capability to automatically upload data when connected to a Wi-Fi environment. Furthermore, the data transmission unit can adjust the frequency and timing of data transmission according to user settings. Furthermore, it incorporates data encryption functionality to ensure the security of data during transmission.

The data analysis unit is located on the cloud server and operates generative AI. This unit analyzes transmitted biometric data using machine learning algorithms. Specifically, the AI performs pattern recognition based on large datasets to detect abnormal values and trends. For example, it estimates stress levels from heart rate variability and detects signs of fever from changes in body temperature. It also analyzes sleep data to evaluate sleep quality and provides specific recommendations for improvement. Furthermore, the AI monitors changes in health status by comparing against historical data and sends alerts to caregivers or medical staff when abnormalities are detected. Specific examples of prompt sentences fed to the generative AI include: “Analyze the care recipient's heart rate data and estimate their stress level,” or “Evaluate sleep quality based on sleep data and propose improvement measures.”

The User Interaction component is implemented as an application on the device, providing advice to caregivers based on the analysis results. This component visually displays the analysis results to enable caregivers to understand them intuitively. For example, if a sudden fluctuation in heart rate is detected, the AI suggests the possibility of stress or physical discomfort and prompts the caregiver to implement relaxation techniques or contact a medical institution. Additionally, if sleep quality is poor, it suggests improvements to the sleep environment or bedtime routines. Caregivers can implement the advice via the app and provide feedback on the results. This feedback is used as data for the AI to learn from, helping it provides more personalized advice. Furthermore, the User Interaction Unit includes a voice assistant function, allowing caregivers to call the AI via voice and receive advice.

Thus, the system according to this embodiment enables caregivers to grasp the health status of care recipients in real time and provide appropriate care by combining a data collection unit, a data transmission unit, a data analysis unit, and a user interaction unit. This is expected to improve the quality of care and reduce the burden on caregivers.

### Implementation Steps

### Step 1: Data Collection

The care recipient wears a wearable device that collects real-time biometric data such as heart rate, body temperature, activity level, sleep patterns, and blood oxygen saturation. This device incorporates sensors including a heart rate sensor, temperature sensor, accelerometer, gyroscope, and optical sensor, which work together to acquire data. For example, the heart rate sensor uses optical technology to detect changes in blood flow and measure heart rate. The temperature sensor measures skin temperature, enabling real-time detection of subtle fluctuations in body temperature. The accelerometer and gyroscope detect the care recipient's movements and posture to measure activity levels. The optical sensor measures blood oxygen saturation to monitor respiratory status.

### Step 2: Data Transmission

Collected data is transmitted to a cloud server via the care recipient's terminal. The terminal is a portable electronic device, such as a smartphone or tablet, which communicates with wearable devices using Bluetooth or Wi-Fi. The data transmission unit temporarily buffers the data and efficiently transmits it when the communication environment is ready. For example, it has a function to automatically upload data when connected to a Wi-Fi environment. It also incorporates data encryption functionality to ensure the security of data during transmission.

### Step 3: Data Analysis

Generative AI operates within the data analysis unit established on the cloud server to analyze transmitted biometric data. This unit analyzes the transmitted biometric data using machine learning algorithms. Specifically, the AI performs pattern recognition based on large datasets to detect abnormal values and trends. For example, it estimates stress levels from heart rate variability and detects signs of fever from changes in body temperature. It also analyzes sleep data to evaluate sleep quality and provides specific recommendations for improvement. Furthermore, the AI monitors changes in health status by comparing against historical data and sends alerts to caregivers or medical staff when abnormalities are detected. Specific examples of prompts fed to the generative AI include: “Analyze the care recipient's heart rate data and estimate their stress level” or “Evaluate sleep quality based on sleep data and propose improvement measures.”

### Step 4: User Interaction

Analysis results are provided to caregivers through the user interaction component, implemented as an application on the device. This component visually displays analysis results, enabling caregivers to understand them intuitively. For example, if a sudden fluctuation in heart rate is detected, the AI suggests the possibility of stress or physical discomfort and prompts the caregiver to implement relaxation techniques or contact a medical institution. Additionally, if sleep quality is poor, it proposes improvements to the sleep environment or bedtime routines. Caregivers can implement the advice via the app and provide feedback on the results. This feedback is utilized as data for the AI to learn from, helping it provides more personalized advice. Furthermore, the User Interaction section features a voice assistant function, allowing caregivers to call upon the AI via voice commands to receive guidance. to provide more personalized advice. Furthermore, the user interaction unit incorporates a voice assistant function, allowing caregivers to call upon the AI via voice commands to receive guidance.

### Specific Use Cases

For example, consider using the present invention's system in a care facility. In this facility, elderly residents wear a wearable device that collects real-time biometric data during daily life, such as heart rate, body temperature, activity levels, sleep patterns, and blood oxygen saturation. The heart rate sensor uses optical technology to detect changes in blood flow and measure heart rate. This allows it to suggest potential stress or poor health if the heart rate is abnormally high even at rest. The temperature sensor measures skin temperature, enabling real-time monitoring of subtle fluctuations in body temperature. This facilitates early detection of fever signs, reducing the risk of infection. Accelerometers and gyroscopes detect an elderly person's movements and posture to measure activity levels. This enables assessment of fall risk and implementation of preventive measures. Optical sensors measure blood oxygen saturation to monitor respiratory status. This allows early detection of signs of respiratory distress and prompts appropriate action.

Collected data is transmitted to a cloud server via terminals within the facility. The terminals communicate with wearable devices using Bluetooth or Wi-Fi, temporarily buffer the data, and efficiently transmit it when communication conditions are favorable. Data is encrypted to ensure security.

The data analysis unit on the cloud server employs generative AI to analyze data using machine learning algorithms. The AI estimates stress levels from heart rate variability and detects signs of fever from changes in body temperature. It also analyzes sleep data to evaluate sleep quality and provides specific recommendations for improvement. Furthermore, the AI monitors changes in health status by comparing data to historical records and sends alerts to care staff if abnormalities are detected. Specific examples of prompt sentences fed to the generative AI include: “Analyze the heart rate data of the elderly and estimate stress levels” or “Evaluate sleep quality based on sleep data and propose improvement measures.”

Analysis results are notified to caregivers' terminals, proposing specific care plans or emergency response measures. For example, if a sudden fluctuation in heart rate is detected, the AI suggests the possibility of stress or physical discomfort, prompting caregivers to implement relaxation techniques or contact medical facilities. If sleep quality is poor, it proposes improvements to the sleep environment or bedtime routines. Care staff can execute the advice via the app and provide feedback on the results. This feedback is utilized as data for the AI to learn from, helping it provides more personalized advice.

Thus, the system of the present invention enables real-time monitoring of the health status of elderly individuals in care facilities, allowing care staff to provide appropriate care. This is expected to improve the quality of care and reduce the burden on care staff. Furthermore, the elderly individuals themselves can check their health status and receive advice to improve their quality of life.

The specific processing unit 290 transmits the results of the specific processing to the smart device 14. On the smart device 14, the control unit 46A instructs the output device 40 to output the results of the specific processing. The microphone 38B acquires audio indicating user input regarding the results of the specific processing. The control unit 46A transmits the audio data indicating the user input acquired by the microphone 38B to the data processing unit 12. At the data processing unit 12, the specific processing unit 290 acquires the audio data.

Data Generation Model 58 is what is known as generative AI (Artificial Intelligence). An example of a data generation model 58 is ChatGPT (registered trademark) (Internet search <URL:https://openai.com/blog/chatgpt>). Data generation model 58 is obtained by performing deep learning on a neural network. Data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58. The data generation model 58 includes AI other than generative AI. AI other than generative AI includes, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such examples. Furthermore, the AI may be an AI agent. Also, when the processing of the aforementioned components is performed by AI, that processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit acquires step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the smart device 14.

### Second Embodiment

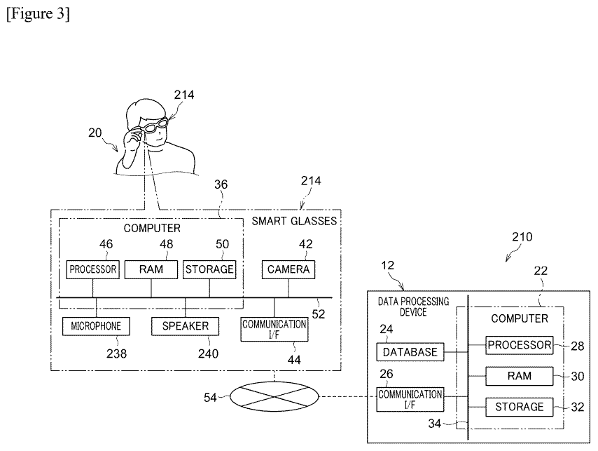

FIG. 3 shows an example configuration of the data processing system 210 according to the second embodiment.

As shown in FIG. 3, the data processing system 210 includes a data processing device 12 and smart glasses 214. An example of the data processing device 12 is a server.

The data processing device 12 includes a computer 22, a database 24, and a communication I/F 26. The computer 22 is an example of a “computer” according to the technology of the present disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 is WAN (Wide Area Network) and/or LAN (Local Area Network) are examples.

Smart glasses 214 include a computer 36, a microphone 238, a speaker 240, a camera 42, and a communication I/F 44. The computer 36 includes a processor 46, RAM 48, and storage 50. Processor 46, RAM 48, and storage 50 are connected to bus 52. Microphone 238, speaker 240, and camera 42 are also connected to bus 52.

Microphone 238 receives voice input from user 20, thereby accepting instructions or other input from user 20. Microphone 238 captures the voice input from user 20 and converts the captured audio into audio data, which it outputs to the processor 46. The speaker 240 outputs audio in accordance with instructions from the processor 46.

Camera 42 is a compact digital camera equipped with an optical system, such as a lens, aperture, and shutter, and an imaging element, such as a CMOS (Complementary Metal-Oxide-Semiconductor) image sensor or a CCD (Charge Coupled Device) image sensor. It captures images of the user's surroundings (e.g., within a field of view equivalent to that of a typical healthy individual).

The communication I/F 44 is connected to the network 54. The communication I/Fs 44 and 26 manage the exchange of various information between processor 46 and processor 28 via network 54. The exchange of various information between processor 46 and processor 28 using communication I/F 44 and 26 is performed in a secure state.

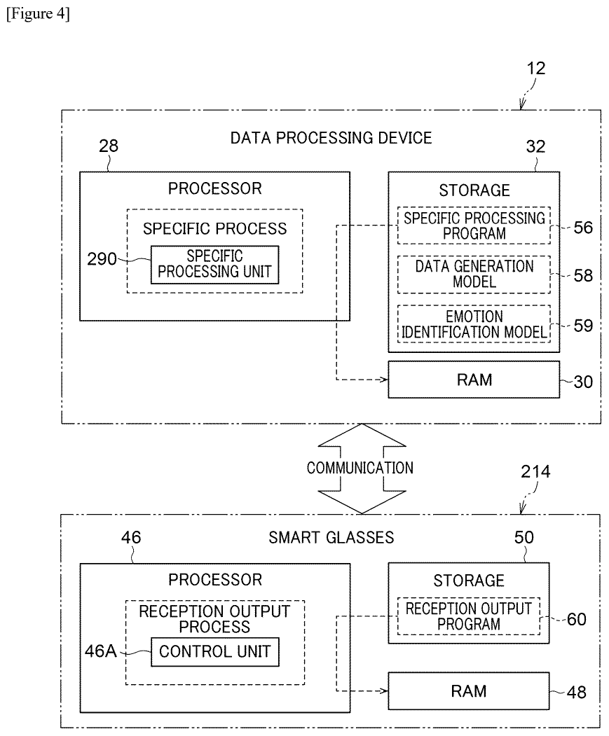

FIG. 4 shows an example of key functions of the data processing device 12 and the smart glasses 214. As shown in FIG. 4, specific processing is performed by the processor 28 in the data processing device 12. The specific processing program 56 is stored in the storage 32.

The specific processing program 56 is an example of a “program” related to the technology of this disclosure. Processor 28 reads the specific processing program 56 from storage 32 and executes the read specific processing program 56 on RAM 30. The specific processing is realized by processor 28 operating as specific processing unit 290 according to the specific processing program 56 executed on RAM 30.

Storage 32 stores a data generation model 58 and an emotion identification model 59. The data generation model 58 and emotion identification model 59 are used by specific processing unit 290. Specific processing unit 290 can estimate a user's emotion using emotion identification model 59 and perform specific processing using the user's emotion. The emotion estimation function (emotion identification function) using the emotion identification model 59 performs various estimations and predictions concerning the user's emotion, including estimation and prediction of the user's emotion, but is not limited to such examples. Furthermore, estimation and prediction of emotion also includes, for example, analysis (parsing) of emotion.

In the smart glasses 214, the processor 46 performs the reception output processing. The reception output program 60 is stored in the storage 50. The processor 46 reads the reception output program 60 from the storage 50 and executes the read reception output program 60 on the RAM 48. The reception output processing is realized by the processor 46 operating as the control unit 46A according to the reception output program 60 executed on the RAM 48. The reception output processing is realized by the processor 46 operating as a control unit 46A according to the reception output program 60 executed on RAM 48. Note that the smart glasses 214 may also have a data generation model 58 and an emotion identification model 59, and can perform processing similar to that of the identification processing unit 290 using these models.

Next, the identification processing performed by the identification processing unit 290 of the data processing device 12 is described. The components of the system described below are implemented by the data processing device 12 and the smart glasses 214. In the following description, the data processing device 12 is referred to as the “server,” and the smart glasses 214 are referred to as the “terminal.”

### Example 1

The flow of the specific processing in Example 1 described in the first embodiment is the same as described above, so the explanation is omitted.

### Application Example 1

The flow of the specific processing in Example 1 described in the first embodiment is the same as described above, so the explanation is omitted.

The specific processing unit 290 transmits the result of the specific processing to the smart glasses 214. In the smart glasses 214, the control unit 46A causes the speaker 240 to output the result of the specific processing. The microphone 238 acquires audio indicating user input regarding the result of the specific processing. The control unit 46A transmits the audio data indicating the user input acquired by the microphone 238 to the data processing device 12. At the data processing device 12, the specific processing unit 290 acquires the audio data.

Data Generation Model 58 is what is known as generative AI (Artificial Intelligence). An example of a data generation model 58 is ChatGPT (registered trademark) (Internet search <URL:https://openai.com/blog/chatgpt>). Data generation model 58 is obtained by performing deep learning on a neural network. Data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such examples. Furthermore, the AI may be an AI agent. Also, when the processing of the aforementioned components is performed by AI, that processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit may acquire step count data using the camera 42 or communication I/F 44 of the smart device 14, and the acquisition unit and collection unit may process the data acquired by the acquisition unit. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the smart glasses 214.

### Third Embodiment

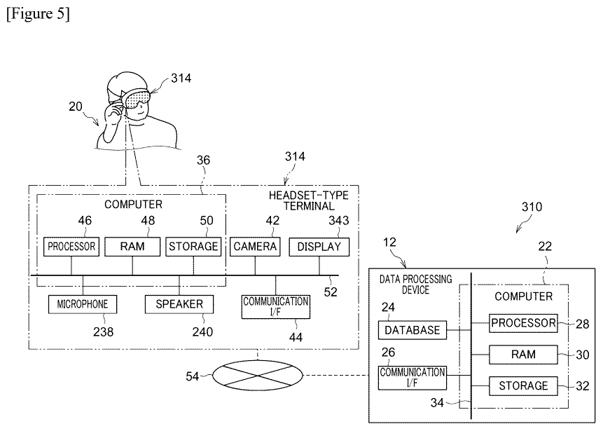

FIG. 5 shows an example configuration of the data processing system 310 according to the third embodiment.

As shown in FIG. 5, the data processing system 310 includes a data processing device 12 and a headset-type terminal 314. An example of the data processing device 12 is a server.

Data processing device 12 includes a computer 22, a database 24, and a communication I/F 26. Computer 22 is an example of a “computer” related to the technology of this disclosure. Computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 include a WAN (Wide Area Network) and/or a LAN (Local Area Network).

The headset-type terminal 314 comprises a computer 36, a microphone 238, a speaker 240, a camera 42, a communication interface 44, and a display 343. The computer 36 includes a processor 46, RAM 48, and storage 50. The processor 46, RAM 48, and storage 50 are connected to a bus 52. The microphone 238, speaker 240, camera 42, and display 343 are also connected to the bus 52.

Microphone 238 receives voice input from user 20 to accept instructions and the like from user 20. Microphone 238 captures the voice input from user 20, converts the captured voice into audio data, and outputs it to processor 46. Speaker 240 outputs audio according to instructions from processor 46.

The camera 42 is a compact digital camera equipped with an optical system, such as a lens, aperture, and shutter, and an imaging element, such as a CMOS (Complementary Metal-Oxide-Semiconductor) image sensor or a CCD (Charge Coupled Device) image sensor. It captures the user's surroundings (e.g., an imaging range defined by a field of view equivalent to that of a typical healthy person).

The communication I/F 44 is connected to the network 54. The communication I/Fs 44 and 26 handle the exchange of various information between processor 46 and processor 28 via network 54. The exchange of various information between processor 46 and processor 28 using communication I/F 44 and 26 is performed in a secure state.

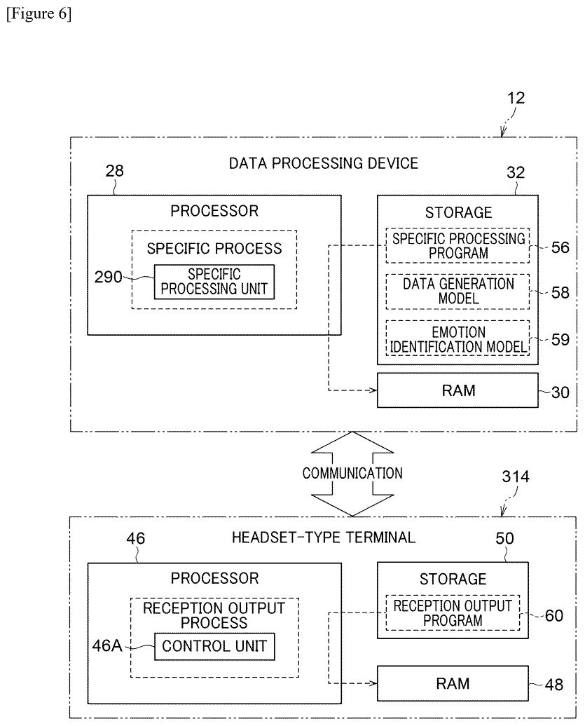

FIG. 6 shows an example of the main functions of the data processing device 12 and the headset-type terminal 314. As shown in FIG. 6, specific processing is performed by the processor 28 in the data processing device 12. The specific processing program 56 is stored in the storage 32.

The specific processing program 56 is an example of a “program” related to the technology of this disclosure. The processor 28 reads the specific processing program 56 from the storage 32 and executes the read specific processing program 56 on the RAM 30. The specific processing is realized by the processor 28 operating as a specific processing unit 290 according to the specific processing program 56 executed on the RAM 30.

Storage 32 stores a data generation model 58 and an emotion identification model 59. The data generation model 58 and the emotion identification model 59 are used by the specific processing unit 290.

In the headset-type terminal 314, reception output processing is performed by the processor 46. The reception output program 60 is stored in the storage 50. Processor 46 reads the reception output program 60 from storage 50 and executes the read reception output program 60 on RAM 48. Reception output processing is achieved by processor 46 operating as control unit 46A according to the reception output program 60 executed on RAM 48.

Next, the specific processing performed by the specific processing unit 290 of the data processing device 12 is described. The various parts of the system described below are implemented by the data processing device 12 and the headset-type terminal 314. In the following description, the data processing device 12 is referred to as the “server,” and the headset-type terminal 314 is referred to as the “terminal.”

### Example 1

The flow of the specific processing is the same as that described in Example 1 of the first embodiment, so the description is omitted.

### Application Example 1

The flow of the specific processing is the same as that described in Example 1 of the first embodiment above; therefore, the description is omitted.

The specific processing unit 290 transmits the result of the specific processing to the headset-type terminal 314. At the headset-type terminal 314, the control unit 46A causes the speaker 240 and the display 343 to output the result of the specific processing. The microphone 238 acquires audio indicating user input regarding the result of the specific processing. The control unit 46A transmits the audio data indicating the user input acquired by the microphone 238 to the data processing device 12. At the data processing device 12, the specific processing unit 290 acquires the audio data.

Data Generation Model 58 is a so-called generative AI (Artificial Intelligence). An example of a data generation model 58 is ChatGPT (registered trademark) (Internet search <URL:https://openai.com/blog/chatgpt>). Data generation model 58 is obtained by performing deep learning on a neural network. Data generation model 58 receives input of a prompt containing instructions, as well as inference data such as audio data representing audio, text data representing text, and image data representing images (e.g., still image data or video data). The data generation model 58 infers based on the input inference data according to the instructions indicated by the prompt and outputs the inference result in one or more data formats such as audio data, text data, and image data. The data generation model 58 may include, for example, text generation AI, image generation AI, multimodal generation AI, etc. Here, inference refers to, for example, analysis, classification, prediction, and/or summarization. The specific processing unit 290 performs the specific processing described above using the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such examples. Furthermore, the AI may be an AI agent. Also, when the processing of the aforementioned parts is performed by AI, that processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit acquires step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12. However, the technology disclosed herein is not limited to this, and specific processing may also be performed by the headset-type terminal 314.

### Fourth Embodiment

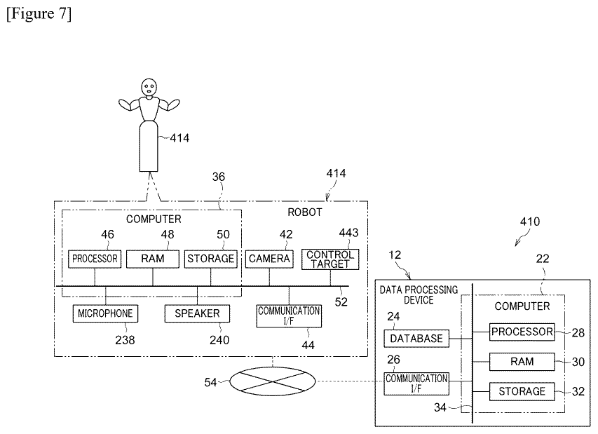

FIG. 7 shows an example configuration of the data processing system 410 according to the fourth embodiment.

As shown in FIG. 7, the data processing system 410 includes a data processing device 12 and a robot 414. An example of the data processing device 12 is a server.

The data processing device 12 includes a computer 22, a database 24, and a communication I/F 26. The computer 22 is an example of a “computer” related to the technology of this disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 include a WAN (Wide Area Network) and/or a LAN (Local Area Network).

Robot 414 includes a computer 36, a microphone 238, a speaker 240, a camera 42, a communication interface 44, and a control target 443. Computer 36 includes a processor 46, RAM 48, and storage 50. Processor 46, RAM 48, and storage 50 are connected to bus 52. Furthermore, microphone 238, speaker 240, camera 42, and controlled object 443 are also connected to bus 52.

Microphone 238 receives voice input from user 20 to accept instructions or other commands. Microphone 238 captures the voice input from user 20, converts the captured voice into audio data, and outputs it to processor 46. Speaker 240 outputs audio in accordance with instructions from processor 46.

The camera 42 is a compact digital camera equipped with an optical system, such as a lens, aperture, and shutter, and an imaging element, such as a CMOS (Complementary Metal-Oxide-Semiconductor) image sensor or a CCD (Charge Coupled Device) image sensor. It captures images of the user's surroundings (e.g., an imaging range defined by a field of view equivalent to that of a typical healthy person).

The communication I/F 44 is connected to the network 54. The communication I/Fs 44 and 26 manage the exchange of various information between processor 46 and processor 28 via network 54. The exchange of various information between processor 46 and processor 28 using communication I/F 44 and 26 is performed in a secure state.

The control target 443 includes a display device, LEDs for the eye section, and motors for driving the arms, hands, legs, etc. The posture and gestures of robot 414 are controlled by controlling the motors for the arms, hands, legs, etc. Part of the robot 414's emotions can be expressed by controlling these motors. Furthermore, the robot 414's facial expressions can also be expressed by controlling the light emission state of the LEDs in its eyes.

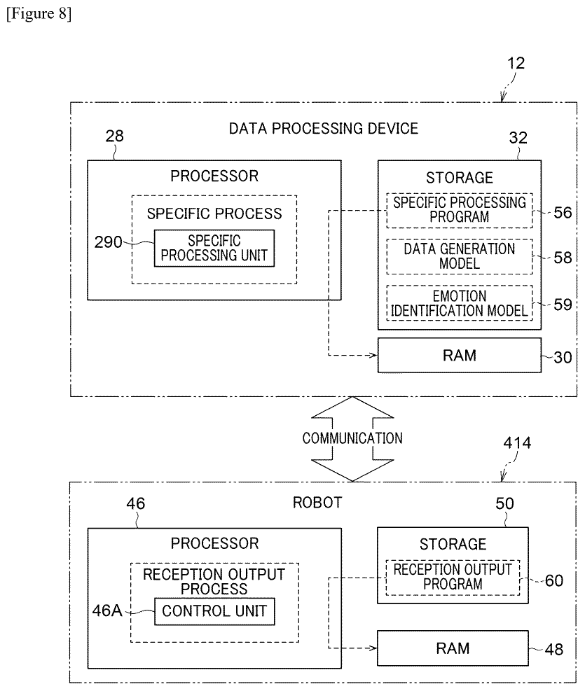

FIG. 8 shows an example of the main functions of the data processing device 12 and the robot 414. As shown in FIG. 8, specific processing is performed by the processor 28 in the data processing device 12. The specific processing program 56 is stored in the storage 32.

The specific processing program 56 is an example of a “program” related to the technology of this disclosure. The processor 28 reads the specific processing program 56 from the storage 32 and executes the read specific processing program 56 on the RAM 30. The specific processing is realized by the processor 28 operating as a specific processing unit 290 according to the specific processing program 56 executed on the RAM 30.

Storage 32 stores a data generation model 58 and an emotion identification model 59. The data generation model 58 and the emotion identification model 59 are used by the specific processing unit 290.

In robot 414, reception output processing is performed by processor 46. Storage 50 stores a reception output program 60. Processor 46 reads the reception output program 60 from storage 50 and executes the read reception output program 60 on RAM 48. Reception output processing is realized by the processor 46 operating as a control unit 46A according to the reception output program 60 executed on RAM 48.

Next, the specific processing performed by the specific processing unit 290 of the data processing device 12 is described. The various parts of the system described below are realized by the data processing device 12 and the robot 414. In the following description, the data processing device 12 is referred to as the “server,” and the robot 414 is referred to as the “terminal.”

### Example 1

The flow of the specific processing is the same as that described in Example 1 of the first embodiment, so the description is omitted.

### Application Example 1

The flow of the specific processing in Example 1 described in the above first embodiment is the same, so the explanation is omitted.

The specific processing unit 290 transmits the result of the specific processing to the robot 414. In the robot 414, the control unit 46A causes the speaker 240 and the control target 443 to output the result of the specific processing. The microphone 238 acquires audio indicating user input regarding the result of the specific processing. The control unit 46A transmits the audio data indicating the user input acquired by the microphone 238 to the data processing device 12. At the data processing device 12, the specific processing unit 290 acquires the audio data.

Data Generation Model 58 is what is known as generative AI (Artificial Intelligence). An example of a data generation model 58 is ChatGPT (registered trademark) (Internet search <URL:https://openai.com/blog/chatgpt>). Data generation model 58 is obtained by performing deep learning on a neural network. Data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), and recurrent neural networks (RNN), generative adversarial networks (GAN), or naive Bayes, among others. The AI may perform various processing operations, but is not limited to such examples. Furthermore, the AI may be an AI agent. Also, when the processing of the aforementioned components is performed by AI, that processing may be performed partially or entirely by AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit acquires step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the robot 414.

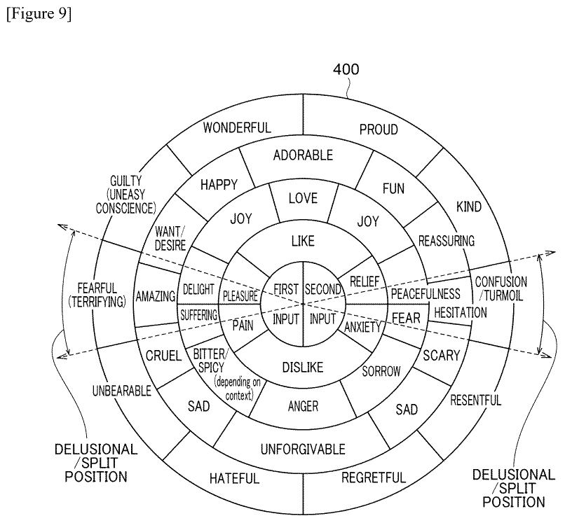

The emotion identification model 59, functioning as an emotion engine, may determine the user's emotion according to a specific mapping. Specifically, the emotion identification model 59 may determine the user's emotion according to an emotion map (see FIG. 9), which is a specific mapping. Furthermore, the emotion identification model 59 may similarly determine the robot's emotion, and the specific processing unit 290 may perform specific processing using the robot's emotion.

FIG. 9 is a diagram showing an emotion map 400 where multiple emotions are mapped. In the emotion map 400, emotions are arranged in concentric circles radiating from the center. Emotions closer to the center of the concentric circles represent more primitive states. Emotions representing states or behaviors arising from mental states are placed further out in the concentric circles. Emotion is a concept encompassing affect and mental states. Generally, emotions generated from reactions occurring within the brain are placed on the left side of the concentric circles. Generally, emotions induced by situational judgment are placed on the right side of the concentric circles. Generally, emotions generated from reactions occurring within the brain and also induced by situational judgment are placed on the upper and lower sides of the concentric circles. Furthermore, the upper part of the concentric circle contains “pleasant” emotions, while the lower part contains “unpleasant” emotions. Thus, the Emotion Map 400 maps multiple emotions based on the structure of their origin, with emotions that tend to occur simultaneously mapped close together.

These emotions are distributed around the 3 o'clock position on Emotion Map 400, typically oscillating between feelings of security and anxiety. In the right half of Emotion Map 400, situational awareness takes precedence over internal sensations, resulting in a calmer impression.

The inner part of the emotion map 400 represents the mind, while the outer part represents behavior. Therefore, the further outward one goes on the emotion map 400, the more visible the emotion becomes (manifesting in behavior).

Here, human emotions are based on various balances, such as posture and blood sugar levels. When these balances deviate from the ideal, it indicates a state of discomfort; when they approach the ideal, it indicates a state of comfort. Similarly, for robots, automobiles, motorcycles, etc., emotions can be created based on various balances, such as posture and battery level. When these balances deviate from the ideal, it indicates a state of discomfort; when they approach the ideal, it indicates a state of comfort. The emotion map is, for example, Dr. Mitsuyoshi's Emotion Map (Based on research on speech emotion recognition and neurophysiological signal analysis of emotions, Tokushima University, Doctoral Dissertation: https://ci.nii.ac.jp/naid/500000375379). The left half of the emotion map displays emotions belonging to the “Reaction” domain, where sensory aspects predominate. The right half of the emotion map displays emotions belonging to the “Situation” domain, where situational awareness is dominant.

The emotion map defines two emotions that promote learning. One is the negative emotion around the center of the “repentance” or “reflection” area on the situation side. That is, when the robot experiences negative emotions like “I never want to feel this way again” or “I don't want to be scolded anymore.” The other is the positive emotion around “desire” on the reaction side. That is, when the robot feels positive emotions like “I want more” or “I want to know more.”

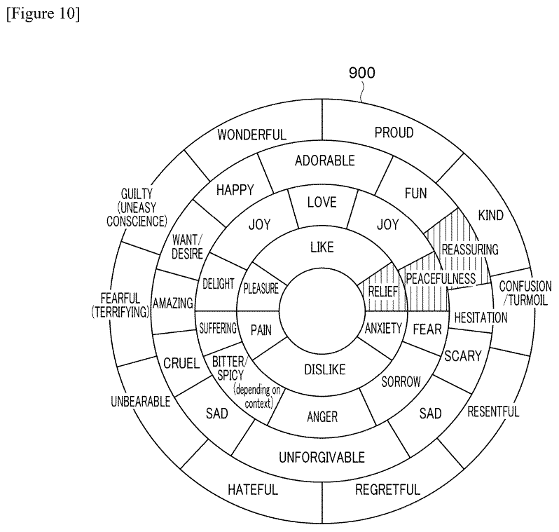

The emotion identification model 59 inputs the user input into a pre-trained neural network, obtains emotion values corresponding to each emotion shown in the emotion map 400, and determines the user's emotion. This neural network is pre-trained based on multiple training data sets, which are combinations of user input and emotion values corresponding to each emotion shown in the emotion map 400. Furthermore, this neural network is trained such that emotions positioned close to each other, as shown in Emotion Map 900 in FIG. 10, have similar values. FIG. 10 illustrates an example where multiple emotions, such as “reassurance,” “tranquility,” and “encouragement,” have similar emotion values.

The above description primarily explains the system of the present disclosure in terms of the functions of the data processing device 12. However, the system of the present disclosure is not necessarily implemented on a server. The system of the present disclosure may be implemented as a general information processing system. For example, the present disclosure may be implemented as a software program operating on a personal computer or as an application operating on a smartphone, etc. The method of the present disclosure may be provided to users in a SaaS (Software as a Service) format.

The above embodiment illustrated an example configuration where specific processing is performed by a single computer 22. However, the technology of this disclosure is not limited thereto. Distributed processing may be performed by multiple computers, including computer 22, for specific processing. For example, data generation model 58 may be provided in an external device of data processing device 12, and data generation corresponding to input data may be performed in said external device. For example, the data generation model 58 may be provided in an external device of the data processing device 12, and data generation corresponding to input data may be performed in said external device.

The above embodiment described a configuration where the specific processing program 56 is stored in the storage 32. However, the technology disclosed herein is not limited to this. For example, the specific processing program 56 may be stored on a portable, computer-readable non-volatile storage medium, such as a USB (Universal Serial Bus) memory. The specific processing program 56 stored on the non-volatile storage medium is installed on the computer 22 of the data processing device 12. The processor 28 executes specific processing according to the specific processing program 56.

Alternatively, the specific processing program 56 may be stored on a storage device, such as a server, connected to the data processing device 12 via the network 54. Upon request from the data processing device 12, the specific processing program 56 may be downloaded and installed on the computer 22.

It should be noted that it is not necessary to store the entire specific processing program 56 on a storage device such as a server connected to the data processing device 12 via the network 54, or to store the entire specific processing program 56 in the storage 32. It is also possible to store only a portion of the specific processing program 56.

Various types of processors can be used as hardware resources to execute the specific processing. Examples of processors include a CPU, which is a general-purpose processor that functions as a hardware resource for executing specific processing by executing software, i.e., programs. Additionally, processors may include dedicated electronic circuits, such as FPGAs (Field-Programmable Gate Array), PLDs (Programmable Logic Device), or ASICs (Application Specific Integrated Circuit), which are processors with circuit configurations specifically designed to execute particular processing. Each processor has memory either built-in or connected, and each processor executes specific processing by using this memory.

The hardware resources for executing specific processing may be comprised of one of these various processors, or may be comprised of a combination of two or more processors of the same or different types (e.g., a combination of multiple FPGAs, or a combination of a CPU and an FPGA). Furthermore, the hardware resources for executing specific processing may be a single processor.

Examples of configurations using a single processor include: First, a configuration where one processor is formed by combining one or more CPUs with software, with this processor functioning as the hardware resource executing specific processing. Second, there is a form using a processor that implements the entire system's functionality, including multiple hardware resources executing specific processing, on a single IC chip, as exemplified by a System-on-a-chip (SoC). Thus, specific processing is implemented as a hardware resource using one or more of the various processors described above.

Furthermore, regarding the hardware structure of these various processors, more specifically, electrical circuits combining circuit elements such as semiconductor devices can be used. Also, the specific processing described above is merely one example. Therefore, it goes without saying that within the scope not deviating from the main purpose, unnecessary steps may be omitted, new steps may be added, or the processing order may be changed.

The above description and illustrations provide a detailed explanation of the aspects pertaining to the technology of this disclosure and represent merely one example of the technology disclosed herein. For example, the explanations regarding the configuration, functions, operations, and effects described above are examples of the configuration, functions, operations, and effects pertaining to the aspects of the technology disclosed herein. Therefore, it goes without saying that within the scope of not deviating from the essence of the technology of this disclosure, unnecessary portions may be deleted, new elements may be added, or replacements may be made to the above-described content and illustrated content. Furthermore, to avoid complexity and facilitate understanding of the part pertaining to the technology of this disclosure, descriptions of technical common knowledge, etc., that are particularly unnecessary for enabling the implementation of the technology of this disclosure have been omitted from the above-described content and illustrated content.

All literature, patent applications, and technical standards described herein are incorporated by reference into this specification to the same extent as if each individual literature, patent application, and technical standard were specifically and individually incorporated by reference.

Regarding the above embodiments, the following is further disclosed.

### Supplementary Note 1

A system comprising: a data collection unit for collecting biometric data from a wearable device; a data transmission unit for transmitting the collected biometric data to a cloud server; a data analysis unit including a generative AI that analyzes the biometric data at the cloud server; and a user interaction unit that provides advice to caregivers based on the analysis results.

A wearable device that measures heart rate, body temperature, activity level, sleep patterns, and blood oxygen concentration in real time, and transmits this data to a cloud server via Bluetooth or Wi-Fi. The cloud server uses machine learning algorithms to recognize patterns in the data, detect abnormal values or trends, and propose specific care plans or emergency response measures to caregivers.

The user interaction unit notifies the caregiver's terminal of the analysis results. When it detects fluctuations in stress levels or a decline in sleep quality, it proposes relaxation methods or sleep environment improvement measures. By receiving feedback from the caregiver on the results of implementing the advice, the AI learns the effectiveness and provides more personalized advice. This characterizes the system described in Supplementary Note 1.

### Explanation of Symbols

10, 210, 310, 410 Data Processing System
12 Data Processing Device
14 Smart Device
214 Smart Glasses
314 Headset-type devices
414 Robot

What is claimed is

1. A biometric monitoring and adaptive intervention system, comprising:
a wearable device configured to be worn on a user's body and comprising:
a heart rate sensor configured to detect blood flow using optical measurement,
a temperature sensor configured to measure skin temperature,
at least one motion sensor comprising an accelerometer or gyroscope configured to detect movement and posture, and
a blood oxygen sensor configured to measure blood oxygen saturation;
a terminal device comprising:
a wireless communication interface configured to communicate with the wearable device via Bluetooth or Wi-Fi,
a display and a speaker configured to output visual and audio information, and
a microphone configured to receive user voice input;
a cloud server comprising:
a communication interface configured to securely receive encrypted biometric data from the terminal device;
a memory storing:
a predefined multidimensional emotion map arranged in concentric regions in which inner regions correspond to internal mental states and outer regions correspond to behavioral expression states, the emotion map including a reaction domain and a situation domain arranged along orthogonal axes; and
a neural network trained using training data associating biometric feature values with emotion values mapped to positions on the emotion map, wherein the training constrains emotion values corresponding to adjacent positions on the emotion map to be similar; and
at least one processor configured to:
extract heart rate variability parameters from inter-beat interval measurements and extract motion and temperature features to generate a biometric feature vector;
input the biometric feature vector to the neural network to generate emotion values corresponding to positions on the emotion map;
determine an emotional coordinate within the emotion map based on the generated emotion values;
determine whether the emotional coordinate falls within a predefined stress-related region or emergency region of the emotion map;
dynamically adjust an anomaly detection threshold for heart rate variability based on a distance between the emotional coordinate and a center of the emotion map;
generate intervention data using a generative AI model conditioned on the emotional coordinate; and
update at least one weighting coefficient used in generating the biometric feature vector based on user feedback indicating effectiveness of the intervention data;
wherein the processor further transmits control data to the wearable device to modify biometric data sampling frequency when the emotional coordinate enters the emergency region.

2. The system of claim 1, wherein the heart rate variability parameters include time-domain dispersion measures and frequency-domain power spectral density components.

3. The system of claim 1, wherein the generative AI model outputs both text guidance displayed on the terminal device and synthesized speech output via the speaker.

4. The system of claim 1, wherein the stress-related region corresponds to an outer lower portion of the concentric emotion map associated with unpleasant behavioral states.

5. The system of claim 1, wherein the processor transmits an alert to a caregiver terminal when the emotional coordinate falls within the emergency region.

6. The system of claim 1, wherein the anomaly detection threshold is increased in sensitivity as the emotional coordinate moves radially outward from the center of the emotion map.

7. A computer-implemented method executed by a cloud server for adaptive biometric monitoring, comprising:
receiving encrypted time-series biometric data collected by a wearable device including heart rate, skin temperature, motion, and blood oxygen saturation data;
extracting inter-beat intervals from the heart rate data and computing heart rate variability features including time-domain and frequency-domain components;
extracting posture and activity features from accelerometer or gyroscope data;
forming a multidimensional biometric feature vector including the heart rate variability features and motion features;
inputting the biometric feature vector into a neural network trained according to a predefined emotion map arranged in concentric regions including a reaction domain and a situation domain;
outputting emotion values corresponding to mapped positions on the emotion map;
determining an emotional coordinate within the emotion map based on the emotion values;
computing a geometric distance between the emotional coordinate and at least one predefined stress or emergency region;
dynamically adjusting a biometric anomaly detection threshold based on the geometric distance; and
generating and transmitting intervention instructions to a terminal device when the adjusted biometric anomaly detection threshold is exceeded.

8. The method of claim 7, further comprising modifying a sampling frequency of the wearable device based on the geometric distance.

9. The method of claim 7, wherein the neural network is trained such that emotion values corresponding to adjacent mapped positions on the emotion map have minimized divergence.

10. The method of claim 7, further comprising receiving user feedback through a voice input at the terminal device and updating a weighting coefficient of the biometric feature vector based on the feedback.

11. The method of claim 7, wherein the intervention instructions include relaxation breathing guidance when the emotional coordinate corresponds to a stress-associated region.

12. The method of claim 7, further comprising transmitting an alert to a caregiver device when the emotional coordinate corresponds to a predefined emergency region.

13. A non-transitory computer-readable medium storing instructions that, when executed by a processor of a cloud server, cause the processor to:
receive encrypted biometric time-series data from a terminal device that communicates with a wearable device;
extract heart rate variability features, motion features, and temperature features from the biometric time-series data;
generate a biometric feature tensor comprising the extracted features;
map the biometric feature tensor to emotion values corresponding to positions on a predefined emotion map arranged in concentric regions including a reaction domain and a situation domain;
determine an emotional coordinate within the emotion map based on the emotion values;
generate intervention content conditioned on the emotional coordinate using a generative AI model; and
update weighting coefficients of the biometric feature tensor using gradient-based updating based on received user feedback indicating effectiveness of the intervention content.

14. The non-transitory computer-readable medium of claim 13, wherein the instructions further cause the processor to modify an anomaly detection sensitivity parameter based on a radial distance of the emotional coordinate from a center of the emotion map.

15. The non-transitory computer-readable medium of claim 13, wherein the intervention content includes multimodal output comprising text, speech, or visual guidance.

16. The non-transitory computer-readable medium of claim 13, wherein the instructions further cause the processor to generate a caregiver alert when the emotional coordinate corresponds to a predefined abnormal state.

17. The non-transitory computer-readable medium of claim 13, wherein the reaction domain corresponds to internally generated emotional states and the situation domain corresponds to externally induced emotional states.

18. The non-transitory computer-readable medium of claim 13, wherein the instructions further cause the processor to adjust a sampling interval of the wearable device when the emotional coordinate moves into an outer behavioral region of the emotion map.
